(19) United States
(12) Patent Application Publication
(10) Pub. No.: US 2017/0224527 A1
(43) Pub. Date: Aug. 10, 2017

(54) Transportable therapy device for the therapeutic treatment of at least one person, in particular of a painful condition of the limb of the person

(71) Applicant: Bernd Zimmer, Neu-Ulm (DE)

(72) Inventor: Konrad Boegelein, Westerheim (DE)

(21) Appl. No.: 15/502,230

(22) PCT Filed: Aug. 7, 2015
(86) PCT No.: PCT/EP2015/068279

§ 371 Date: Feb. 7, 2017

(30) Foreign Application Priority Data
Aug. 8, 2014 (DE) 10 2014 215 795.1

(51) Int. Cl. A61F 7/00

(52) U.S. Cl. CPC A61F 7/0053; A61F 7/007; A61F 2007/0096; A61F 2007/0075; A61F 2007/0031; A61F 2007/0032; A61F 2007/0035; A61F 2007/0045; A61F 2007/0043; A61F 2007/0042; A61F 2007/0057

(57) ABSTRACT

The present invention relates to a transportable therapy device for the therapeutic treatment of at least one person, in particular of a painful condition of at least one limb of the person, comprising a box-like housing which encloses at least one cold chamber which is only accessible from outside via at least one opening, the at least one opening being formed and arranged in the housing in such a way to introduce a limb, which is to be treated, into the cold chamber, comprising at least one cooling means which is thermally coupled in such a way to the cold chamber to cool the cold chamber and is configured in such a way to generate in the cold chamber a predetermined temperature for the therapeutic treatment of the limb which has been introduced, in particular for the therapeutic treatment of a painful condition of the limb which has been introduced.

### FIELD OF THE INVENTION

The invention relates to a transportable therapy device for the therapeutic treatment of at least one person, in particular of a painful condition of the limb of the person.

### TECHNICAL BACKGROUND

Hitherto, to treat people with cold (cryotherapy), in addition to cold packs, on the one hand devices which are operated with liquid gases, for example nitrogen or are operated by means of compressors and evaporators are known for local cryotherapy and on the other hand cold chambers which are fixedly integrated into buildings are known for whole body treatment. Cryotherapy is a method which is mainly for the treatment of pain, both of an inflammatory, rheumatoid origin and also due to other causes, for example following injury. A cold chamber is disclosed, for example, in DE 34 41 091 A. In this respect, to carry out a whole body treatment, a patient's body has to be exposed to a very low temperature for a specific time. Accordingly, a cold chamber having a treatment room for at least one person and a sluice room is provided. A heat exchanger unit comprising an evaporator is provided in the treatment room, as well as for the smaller transportable devices to generate cold air/cold gas for the local treatment of individual body parts.

Cold chambers of this type suffer from the disadvantage that the construction thereof is complex and expensive, and they also have a high energy demand.

### SUMMARY OF THE INVENTION

In view of the above, the object of the invention is to provide an improved therapy device which in particular allows the therapeutic treatment of individual limbs of a person and which can be easily used, for example in any location, in particular also within the home of the person to be treated.

This object is achieved according to the invention by a therapy device having the features of claim 1.

According thereto, a transportable therapy device is provided for the therapeutic treatment of at least one person, in particular of a painful condition of a limb of the person, the device having a box-like housing which encloses at least one cold chamber which is only accessible from outside via at least one opening, the at least one opening being formed and arranged in the housing in order to introduce a limb, which is to be treated, into the cold chamber, having at least one cooling means which is thermally coupled in such a way to the cold chamber to cool said cold chamber and is configured in such a way to generate in the cold chamber a predetermined temperature for the therapeutic treatment of the limb which has been introduced, in particular for the therapeutic treatment of a painful condition of the limb which has been introduced.

In this manner, a therapy device is provided which can be transported to the person to be treated and can thus be used in the person's location. Furthermore, the limb can be effectively cooled in the cold chamber and can be treated thereby, without air being able to undesirably escape outwards from the cold chamber or without air being able to penetrate inside the cold chamber from the surroundings, in that the housing with the limb, introduced into the cold chamber of the housing, is sealed off.

The fundamental understanding/idea of the present invention is to provide a therapy device, in which only one or a plurality of individual limbs of this person are treated by the therapy device. As a result, the therapy device can accordingly be of a small and transportable construction.

Advantageous embodiments and developments of the invention are set out in the subclaims and in the description with reference to the drawings.

In an embodiment of the invention, at least one heating means is provided in the housing to supply, in particular to temporarily supply heat into the cold chamber. Consequently, the person's limb, received in the cold chamber, can be, for example alternately cooled and heated, for example to stimulate metabolism, blood circulation and an immune response. According to a further embodiment of the invention, the cooling means has a fan to cool the air in the cold chamber by circulation. This measure provides an even more effective cooling in the interior of the cold chamber.

According to a further embodiment of the invention, the at least one cooling means, at least one fan and/or at least one heating means is configured to be controllable and/or regulatable by an individual or a common control means. The at least one cooling means, at least one fan and/or at least one heating means can be controlled and/or regulated such that a predetermined temperature or a predetermined temperature profile can be set in the cold chamber. This has the advantage that a temperature or a temperature profile can be set, for example subject to the intended therapy, to the person to be treated and/or to the limb present in the cold chamber, etc. Furthermore, a cold chamber in which the skin can be cooled to a temperature range of between, for example 15C to 25° C., has the advantage that the cold receptors in the skin of the limb can be stimulated and consequently the discharge frequency of pain fibres of the limb can be reduced via central nervous reflexes. In addition, the person to be treated does not perceive this temperature range as being unpleasant, in particular as being too cold, and freezing does not occur.

In a further embodiment of the invention, a temperature sensor means is provided to determine the temperature in the cold chamber. The control means can control and/or regulate the temperature or the temperature profile in the cold chamber subject to the temperature which is detected by the temperature sensor means. In this respect, the temperature sensor means has at least one temperature sensor.

In another embodiment of the invention, the cooling means has at least one Peltier element, at least one metal plate, at least one freezer battery, a cooling gel, ice cubes and/or at least one block of ice. Here, the advantage of a Peltier element is that it can be connected to a socket by a power plug. A metal plate, a freezer battery, a cooling gel, ice cubes and a block of ice can, in turn, be cooled or frozen in situ in a freezer belonging to the person to be treated, to subsequently be used as a cooling means in the therapy device.

According to an embodiment of the invention, the cooling means is configured with cooling lamellae or is coupled thereto on at least one side facing the cold chamber. The advantage of the cooling lamellae or cooling ribs is that they increase the surface area of the cooling means and consequently improve the transfer of heat to the surroundings and thus improve the cooling of the cold chamber.

In another embodiment of the invention, at least one guide plate is provided in the cold chamber of the housing. The guide plate can specifically direct an air flow from the ventilator means in the cold chamber, for example in order to distribute the air in the cold chamber. Furthermore, the at least one guide plate can direct the air flow from the ventilator means along at least one wall of the housing and/or directly onto the limb which has been received in the housing.

In a further embodiment of the invention, the opening has a seal and/or a cover to seal the opening when the limb has been introduced into the opening. The advantage of the cover is that the limb does not have to rest tightly against the opening in order to outwardly tightly seal the housing. Instead, the cover prevents air from penetrating inside the cold chamber from the surroundings and prevents air from undesirably escaping from the cold chamber into the surroundings.

In another embodiment according to the invention, at least one side wall, including for example the front wall and the back wall, is configured such that it folds open in order to introduce the limb into the cold chamber of the housing when the side wall has been folded open. As a result, for example it is very easy for a foot to be introduced into the cold chamber. Furthermore, the side wall can be pulled back into a closed position, for example by a strap attached to the housing, which is particularly convenient for older people and for people with restricted mobility.

According to an embodiment of the invention, the therapy device has a support for the limb which can be received in the cold chamber, which enhances the comfort of the person to be treated. The support can be, for example a lattice. Such a lattice allows the throughflow of air. Moreover, the support can be fitted in the housing in a fixed or removable manner.

In another embodiment of the invention, the housing has a mount for inserting the at least one cooling means. For this purpose, a top cover, a side part or side wall, or a bottom of the housing can be configured in a removable manner in order to insert the cooling means into the mount in the housing. The cooling means can be easily replaced through the removable top cover, side part or side wall, or bottom.

In another embodiment of the invention, the housing has at least two openings for the passage of a limb to receive a part of the limb in the cold chamber of the housing. As a result, for example an elbow, a wrist or a knee can be treated in the therapy device, without the entire arm or the entire leg having to be received in the therapy device. The at least two openings can preferably be provided in opposite sides, for example in the front wall and rear wall, in the bottom and top cover etc., including opposite and mutually adjacent sides, for example the bottom and front wall etc. of the housing. The at least two openings can be provided in particular in a side wall, including a front wall and a rear wall, a top cover and/or a bottom of the housing, depending on which limb is to be guided through, for example an arm or a leg can be stretched out or bent and guided through the housing. The openings can be provided in the housing accordingly.

According to a further embodiment of the invention, the housing is provided outside on its bottom with a rocker element for rocking the therapy device from side to side. Consequently, the limb, for example a foot can be moved during the treatment and does not have to be kept in a predetermined position. This enhances the comfort of the person to be treated.

In another embodiment of the invention, the housing and the cold chamber thereof have a bulge for receiving the limb or limbs. The bulge can provide a greater freedom of movement in the housing for the person to be treated, for example when arms as limbs are introduced into the cold chamber through openings in the opposite side walls of the housing.

In a further embodiment of the invention, the housing is configured such that it is outwardly insulated and consists, for example of an insulation, for example of foamed polystyrene and/or of at least one plastics shell. The advantage of foamed polystyrene and plastics is that they are light materials. Consequently, the therapy device can be configured to be particularly light and is accordingly easy to transport.

According to an embodiment of the invention, at least one top cover and/or one side wall of the housing is configured to be transparent. In this respect, for insulation purposes, for example the top cover and/or the side wall can be produced from a transparent plastics material or transparent glass. If the top cover and/or the side wall are configured with double walls, a gas, for example air can be provided as additional insulation. Furthermore, it is also possible to configure the top cover and/or the side wall in an inflatable manner, in which case the top cover and/or the side wall have an inflatable, preferably a transparent plastics sheath. By virtue of the transparent configuration of at least the top cover and/or of a side wall, it is possible for the limb, received in the housing, to be checked, for the position thereof to be corrected and/or for movements and exercises to be initiated for therapeutic purposes.

In an embodiment of the invention, the air flow, generated by the at least one fan, is strong enough or can be adjusted to be strong enough to cool the limb to be treated by the wind chill effect, in particular air which is close to the skin being removed convectively and the evaporation rate increasing as an accompanying factor, so that the equalisation of the surface temperature of the limb received in the housing with the ambient temperature of the air in the housing accelerates. The skin of the limb received in the housing can be cooled to a temperature range of between, for example, 15° C. to 25° C. The biological effectiveness, i.e. the pain inhibition is improved thereby, without lowering the air temperature in a painful or harmful area.

The above embodiments and developments can be combined together in any appropriate manner. Further possible embodiments, developments and implementations of the invention also include combinations, which have not been explicitly mentioned, of features of the invention which have been previously described or which will be described in the following with respect to the embodiments. In particular, a person skilled in the art will also add individual aspects as improvements or supplements to the respective basic form of the present invention.

### SUMMARY OF THE DRAWINGS

In the following, the present invention will be described in more detail based on the embodiments shown in the figures of the drawings, in which:

FIG. 1 is a perspective view of a transportable therapy device according to a first embodiment of the invention, the therapy device being closed;

FIG. 2 is a perspective view of a transportable therapy device according to a second embodiment of the invention, the therapy device being closed; and

FIG. 3 is a perspective view of a transportable therapy device according to a variant of the second embodiment of the invention, the therapy device being shown in an open position.

The accompanying drawings are to provide a broader understanding of the embodiments of the invention. They illustrate embodiments and, together with the description, serve to explain principles and concepts of the invention. Other embodiments and many of the mentioned advantages will become apparent from looking at the drawings. The elements of the drawings have not necessarily been shown true-to-scale relative to one another.

In the figures of the drawings, identical, functionally identical and identically operating elements, features and components have been respectively provided with the same reference numerals, unless indicate otherwise.

### DESCRIPTION OF EMBODIMENTS

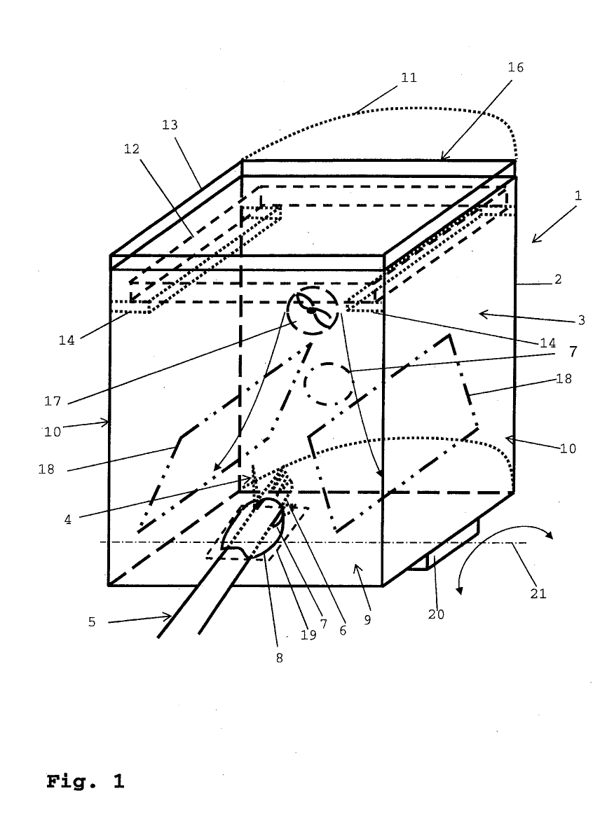

FIG. 1 is a purely schematic and greatly simplified perspective view of a transportable therapy device 1 for the treatment of painful conditions according to an embodiment of the invention. In this case, the therapy device 1 is configured as a cold box 1.

The therapy device 1 has a housing 2 which, inside, forms a cold chamber 3. At least one limb 4 to be treated, for example a hand 6, of a person 5, as shown in the embodiment in FIG. 1, is received and cooled in the cold chamber 3. Instead of a hand 6, it is equally possible, for example, for a foot or an elbow etc. as a limb to be introduced into the cold chamber 3.

In order to introduce the limb 4, the housing 2 has an opening 7 through which the limb 4 can be introduced into the cold chamber 3 of the housing 2. The opening 7 is closed or sealed as a result of introducing the limb 4, preferably being closed or sealed in an airtight manner or in a substantially airtight manner. This has the advantage that, for example cool air from the cold chamber 3 cannot undesirably pass outside and conversely, air, in particular warm air, cannot undesirably pass into the cold chamber 3 from outside. If instead of a hand 6 or a foot, for example a person's knee or elbow is to be treated, a further opening (not shown) can be provided in the housing 2, for example in the opposite or opposite and adjacent side, to pass the arm or leg through. The openings are also sealed by the limb, here by the arm or leg. In this way, the knee or elbow arranged in the cold chamber 3 can be treated therein.

The opening 7 in the cold chamber 3 in the housing 2 optionally also has at least one seal which rests tightly, for example in an airtight manner or in a substantially airtight manner on the limb which has been introduced into the opening. A seal consisting of rubber, for example at least one rubber lip, and/or consisting of foam etc. can be provided for example as the seal.

In addition or alternatively, the opening 7 can also be provided with a cover 8, for example consisting of rubber or woven fabric, preferably an air-impermeable woven fabric etc., through which a limb 4, such as a hand 6, can be guided and can be introduced into the cold chamber 3 through the opening 7. A cover 8 of this type is shown by way of example in FIG. 1. The advantage of the cover 8 is that the opening 7 can be configured in any size and in any shape, since the opening 7 is sealed by the cover 8 and the limb 4 does not have to rest closely on the inner circumference of the opening 7 to prevent air from undesirably entering into and escaping from the cold chamber 3.

The respective cover 8 can be tied down on the limb 4, for example by means of an elastic band or tapes etc., so that air cannot undesirably escape from inside through the cover 8 out of the cold chamber 3 or air cannot undesirably pass from outside via the cover 8 into the cold chamber 3.

Furthermore, the housing 2 and the cold chamber 3 thereof can be configured such that they bulge outwards, for example, as indicated by a dotted line in the embodiment. In this respect, for example instead of an opening 7 in the front 9 of the housing 2, as in the embodiment in FIG. 1, it is also possible for example to respectively provide an opening in the opposite side walls 10 of the housing 2 to introduce the hands from the side into the cold chamber 3 of the housing 2. The bulge 11 of the housing 2 and of the cold chamber 3 thereof, as indicated by a dotted line in FIG. 1, can provide an additional free space for positioning the hands 6 or arms in the cold chamber 3 and this further enhances the comfort for a person 5 who is to be treated.

As previously stated, the therapy device 1 is used to treat pain in limbs 4. In addition to other types of pain, this type of pain in a limb 4 is in particular nociceptor pain. This pain occurs due to the stimulation of pain receptors, known as nociceptors, due to an imminent or sustained injury to the body tissue. This injury to the body tissue can be inflammatory, traumatic, ischemic or tumorous and/or can occur or can be intensified by a sensitisation of the nociceptors. The person becomes aware of the pain due to the conveying of the impulses of the pain receptors to the central nervous system. An inflammatory injury of the body tissue occurs in arthritis, for example. A traumatic injury of the body tissue occurs, for example in a malalignment of joints or in a bone deformation and furthermore can result in osteoarthritis. Osteoarthritis can result, for example from inflammation of a joint or from arthritis and can also be accompanied by an overload-related inflammation reaction, and also by effusion.

Using the therapy device 1, it is possible to achieve a prophylaxis and to mitigate acute swelling and inflammation processes, inter alia for:
blunt trauma,
distortion, contusion
arthritis, polyarthritis
osteoarthritis, polyosteoarthritis
activated osteoarthritis
chronic joint rheumatism
acute flare-up of rheumatism
Psoriasis arthropathica
gout arthropathy
osteoarthritis/arthritis, for example in the hands, fingers, feet, toes
activated osteoarthritis in malalignments and deformities, for example in the feet
tendovaginitis
tendonitis and tendopathy
insertion tendonitis and insertion tendopathy.

In particular, the therapy device 1 can be used to treat inflammatory-rheumatoid disorders, such as arthritis, collagenoses (connective tissue disorders), vasculitides (inflammatory vessel disorders), degenerative or wear-related and/or rheumatic diseases, such as osteoarthritis, metabolic disorders which accompany rheumatic complaints, such as gout, and rheumatic diseases and pain syndromes of the soft tissue, such as myofascial diseases, fibromyalgia (non-inflammatory soft tissue rheumatism), polymyalgia rheumatica (inflammatory soft tissue rheumatism) and polymyositis (inflammation of the muscle tissue). The above-mentioned list is merely an example and is not finite.

Thermoreceptors, here cold receptors in a person's skin can be stimulated painlessly by the cooling effect generated in the cold chamber 3 of the therapy device 1. In the case of thermoreceptors, a distinction is made between cold receptors and warm receptors. Cold receptors of the skin respond at a temperature range of between 15° C. to 25° C. However, at skin temperatures of below 15° C., the cold receptors are increasingly suppressed; instead the pain fibres are activated.

As a result of repeatedly stimulating the cold receptors by exposing the limb 4 in the cold chamber 3 to a skin temperature within a range of, for example 15° C. to 25°, for a treatment duration ranging, for example, between at least 30 minutes and 50 minutes, and with a repetition frequency of, for example, at least twice to three times per week, the discharge frequency of pain fibres of the limb 4 can be reduced and the central pain inhibition can be activated. Consequently, in turn, the patient feels less pain and possibly requires less pain relief.

The therapy device 1 has a cooling means 12 to cool the limb 4, to be treated, in the cold chamber 3. The cold chamber 3 is cooled by the cooling means 12. In this respect, for example at least one Peltier element, at least one metal plate, at least one freezer battery, a cooling gel, ice cubes and/or at least one block of ice etc. can be used as the cooling means 12. The metal plate and the cooling gel can be cooled previously, for example in a freezer compartment of a refrigerator, or a block of ice or the ice cubes can be frozen in the freezer compartment and then introduced into the housing 2 to cool the cold chamber 3. A power plug of the Peltier element can be connected to a socket, for example in situ in the location of the person 5 to be treated.

The invention is not restricted to the examples mentioned for cooling means 12. Any other cooling means 12 can be provided which is suitable for use in the transportable therapy device 1.

The cooling means 12 in the embodiment in FIG. 1 is arranged, for example, above the cold chamber 3. However, the cooling means 12 can also be provided below the cold chamber or on one of the side walls 10 of the cold chamber 3 or of the housing 2, depending on the function and purpose of use. The front wall and the rear wall of the housing also belong to the side walls 10, as they also form a side wall of the housing 2.

In order to insert the cooling means 12, for example a metal plate, in the embodiment in FIG. 1, the housing 2 has a removable top cover 13 which is removed to introduce the cooling means 12 into a corresponding mount 14 in the housing 2. On a lower side or on the side facing the cold chamber 3, the mount 14 or the cooling means 12 itself can optionally also be provided with cooling lamellae or cooling ribs (not shown). Additionally or alternatively to the removable top cover 13, a removable bottom can be provided, for inserting a cooling means.

In the case of blocks of ice, ice cubes or a cooling gel as the cooling means 12, these are received, for example in a container (not shown) and are introduced with the container into the mount 14 in the housing 2. Likewise, the container can optionally also be provided with cooling lamellae or cooling ribs on a lower side or on the side facing the cold chamber 3. Following the treatment of the limb 4, the liquefied blocks of ice or ice cubes and the cooling gel can be easily removed again from the housing 2 with the container and reused or discarded.

In a further alternative embodiment, at least one side of the housing 2 can be provided with a drawer (not shown) to cool a side wall or side, the bottom or the underside, the top cover or the upper side, the front side and/or the rear side of the cold chamber 3.

In a further embodiment of the invention, the cooling means 12, for example a Peltier element can also be fixedly installed in the housing 2, for example it can be screwed onto a wall of the housing 2. Likewise, the Peltier element can optionally also be coupled to cooling lamellae on its underside or on the side facing the cold chamber 3.

The invention is not restricted to the aforementioned examples of arranging the cooling means 12 in the therapy device 1.

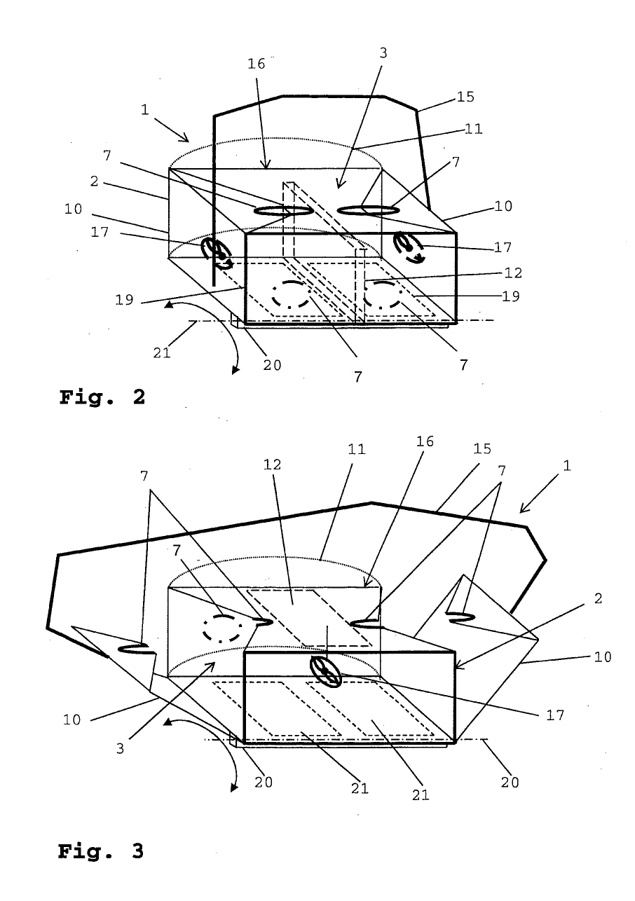

In addition or as an alternative to the cooling means 12, at least one ventilator means 17, configured as a fan, is arranged in the cold chamber 3. A cooling flow of air can be produced by the ventilator means 17 due to the so-called wind chill effect. In this respect, the air flow can be adjusted, for example so that it is strong enough to cool a limb 4 to be treated, but is not so strong that it produces a mechanical, i.e. unpleasant tactile wind stimulus. The at least one ventilator means 17 can be arranged in the housing 2 above the limb 4 to be treated, as shown in FIG. 1, on the side of the limb 17 to be treated, as shown in the following FIGS. 2 and 3, between two limbs, to be treated, as shown in the following FIG. 3, below the limb to be treated and/or in front of the limb 4 to be treated. However, the invention is not restricted to the illustrated or mentioned examples of the at least one ventilator means 17. The at least one ventilator means 17 can be arranged in the housing 2 in any desired position in order to treat the limb which has been introduced.

The wind chill or wind chill effect describes the difference between the measured air temperature and the biologically effective temperature, subject to the wind speed. The wind chill effect is caused by the convective removal of air which is close to the skin and is thus relatively warm. In association therewith, the evaporation rate increases. The energy required for the phase transition of the water is removed by heat conduction out of the surface of the body and cools the body surface accordingly. Therefore, the effect of the wind is to accelerate the equalisation of the surface temperature of the body with the ambient air temperature, which is perceived by people as being cooling. According to the invention, the air flow generated by the at least one ventilator means 17 is preferably strong enough or can be adjusted to be strong enough to cool the limb 4, to be treated, by the wind chill effect, by the convective removal of air close to the skin and by an associated increase in the evaporation rate, so that the equalisation of the surface temperature of the limb, introduced into or passed through the housing 2, with the ambient air temperature in the housing 2 or in the cold chamber 3 thereof is accelerated.

The following empirical formula, valid since November 2001, is used to calculate the so-called wind chill temperature WTC. The empirical formula in the form of a numerical equation for calculating the wind chill using metric units and a wind speed, measured at a height of 10 metres above ground level is as follows:

WCT=13.12+0.6215·Tₐ−11.37·v^{0.16}+0.3965·Tₐ·v^{0.16}
WCT—wind chill temperature in degrees Celsius
Tₐ—air temperature in degrees Celsius
v—wind speed in kilometres per hour

In addition to the generation of a cooling air flow by the ventilator means 17 based on the previously described wind chill effect, additionally or alternatively the ventilator means 17 can be used to distribute the cool air generated by the cooling means 12. In this respect, the air flow can be directed, for example onto the limb 4, and can be optionally guided by at least one guide plate 18, as shown in the embodiment in FIG. 1. Likewise, additionally or alternatively, instead of being guided directly onto the limb 4, the air flow can also be guided according to the invention along the side walls, the bottom and/or the top cover of the cold chamber 3. For this purpose, one or more guide plates (not shown) can optionally also be provided in the cold chamber 3. In this respect, the guide plates 18 can be arranged inside the housing 2 on the side of the limb 4, which has been introduced into or passed through the housing 2, as shown in FIG. 1, above, below, in front of and/or behind said limb, to guide the air flow along at least one wall of the housing, for example a side wall, including the front wall and rear wall, of the bottom and/or of the top cover of the housing 2 and/or to guide the air flow directly onto the limb 4 to be treated. The cooling means 12, optionally a support 19 which is provided and is described in the following, and the at least one guide plate 18 are arranged relative to one another such that an air flow can be directed in an appropriate manner without being obstructed by the cooling means 12 or, conversely, without obstructing the cooling means 12. The same applies to the support 19, if provided.

The support 19 for supporting the limb 4, introduced into the cold chamber 3, can optionally also be provided in the cold chamber 3. The advantage of such a support 19 is that it allows a painless or almost painless positioning and also improves the comfort for a person who is to be treated. In this respect, the support 19 can be, for example a lattice. Such a lattice has the advantage that air can flow through it.

Furthermore, in a further embodiment of the invention, the housing 2 can optionally also have an external rocker element 20 on the bottom thereof. As shown in FIG. 1, the rocker element 20 can have, for example an angular cross section, in particular a rectangular or square cross section, and also a semicircular cross section. However, the invention is not restricted to these cross-sectional shapes. The rocker element can be configured with any cross-sectional shape capable of allowing a rocking movement. In the embodiment in FIG. 1, the rocker element 20 rocks backwards and forwards about its longitudinal axis 21, as indicated by the double arrow. The advantage of the rocker element 20 is that the therapy device 1 can rock backwards and forwards and/or from side to side. As a result, for example in the case of a foot as the limb to be treated, the position of the foot can be changed which enhances the comfort of the person 5 to be treated. In particular, the leg and the adjoining foot do not have to be kept in a particular angled position. Instead, the position of the leg can be varied by the rocker element 20 and furthermore, the therapy device 1 can also be moved more easily, if required. The position, achieved thereby, of the therapy device can also be fixed, as required.

In a further embodiment, in addition to the cooling means 12 and/or to the ventilator means 17, it is also optionally possible to provide a heating means (not shown) for supplying heat into the cold chamber 3. A hot air generator similar to a hair dryer, etc, can be used for example as the heating means. At the same time, for reasons of energy efficiency, the cold generating device can also be temporarily sealed off. In this way, the cold chamber 3 can be alternately cooled or heated, for example in order to stimulate the blood circulation or the metabolic rate of the limb 4 and/or to stimulate the warm receptors of the limb 4. The purpose of the stimulation lies, for example in a “vessel training” by these alternating stimuli, by which vessel training the vegetative control of the vascular muscles can be influenced and thus the blood circulation can be improved. The ability of the vessels to dilate is primarily promoted, since pain results in a contraction and the blood circulation is restricted thereby. Tissue damage which results due to a persistent lack of blood circulation in the case of chronic pain is thereby avoided.

The cooling means 12, the ventilator means 17 and/or the heating means are preferably configured to be controllable and/or regulatable by an individual or by a common control means (not shown). In this respect, the cooling means 12, the ventilator means 17 and/or the heating means can be controlled and/or regulated, for example in respect of their actuation duration, subject to a temperature to be set in the cold chamber 3, subject to a temperature profile to be set in the cold chamber 3, etc. Furthermore, a temperature sensor means (not shown) for determining the temperature in the cold chamber 3 can be provided. The control means can control and/or regulate the cooling means 12, the ventilator means 17 and/or the heating means, subject to the temperature detected by the temperature sensor means. In this way, a constant, predetermined temperature or a predetermined temperature profile can be set in the cold chamber 3.

In the embodiment shown in FIG. 1, an opening 7 is shown for inserting a hand 6 into the cold chamber 3 of the therapy device 1. Instead of one opening 7, the therapy device 1 can also have two openings, for example in one side, as shown in the following FIG. 2, for inserting both hands or, for example both feet into the cold chamber 3 through the respectively associated opening.

Likewise, in further embodiments of the invention, the therapy device 1 can also have at least one opening 7 for passing a limb 4 through, in respectively opposite sides, as previously described. In this way, for example at least one leg or one arm can be inserted into and passed through the therapy device 1, for example in order to position and treat the knee or elbow in the cold chamber 3 of the therapy device 1. As previously described, the respective opening 7 can also be sealed by a seal and/or can be provided with a cover 8. In order to pass a limb 4 through, in the embodiment shown in FIG. 1, the at least one additional opening 7 can be provided in the opposite bottom of the housing 2, as indicated in FIG. 2 by a dash-dot line, in the opposite top cover, in one of the opposite side walls, including the front wall and rear wall, as indicated in FIG. 1 by a dash-dot line. Depending on whether the further opening 7 is provided in the bottom, in the top cover, in one of the side walls of the housing 2 in the embodiment in FIG. 1 or in the following FIGS. 2 and 3, the arrangement of the guide plate or guide plates 18, of the support 19 and of the cooling means 12 is adapted appropriately to provide a treatment of the part of the limb 4, received in the housing 2 and in the cold chamber 3 thereof, such as a knee or elbow etc. The support 19 can also be omitted, if desired, or for example in addition to being fixedly installed, it can also be configured to be removable. This type of arrangement of at least one further opening 7 for guiding a limb 4 through and for treating part of the limb 4, such as the elbow or knee etc., can also be provided in the further embodiments shown in FIGS. 2 and 3. For example, in the following FIGS. 2 and 3, a further opening 7 can be respectively provided in the bottom of the housing 2, as indicated in FIG. 2 by a dash-dot line. In this case, the associated support 19 can be omitted or can be removable, for example. In this way, in the following FIGS. 2 and 3, for example only the respective ankle or for example only the calf can be received in the housing 2 or cold chamber 3. However, the invention is not restricted to this example. In the embodiments shown in FIGS. 1 to 3, each limb 4 or every part of a limb 4 can be introduced or passed through and the therapy device can be accordingly adapted thereto, as required.

FIG. 2 is a purely schematic and greatly simplified perspective view of a second embodiment of a therapy device 1 according to the invention. The therapy device 1 is shown in a closed position.

The therapy device 1 according to FIG. 2 merely differs from the therapy device 1 according to FIG. 1 by the provision of two openings 7 in the upper side 16 or top cover of the housing 2 and of its cold chamber 3 for introducing limbs, for example feet, into the cold chamber 3 of the housing 2. Furthermore, the therapy device 1 according to FIG. 2 differs from the therapy device 1 according to FIG. 1 in that the side walls 10 of the housing 2 are configured to be folding and moreover ventilator means 17 are provided on the inside of the side walls 10. A further difference is that the cooling means 12 is arranged as a dividing wall or partition wall in the cold chamber 3 of the housing 2. Otherwise, with regard to the configuration of the therapy device 1 according to the second embodiment of the invention, reference is made to the description relating to FIG. 1 to save unnecessary repetition.

As shown in the embodiment in FIG. 2, it is very easy for limbs, such as feet, to be introduced from the side into the cold chamber 3 of the housing 2 by means of the folding side walls 10. Using a strap 15 for example, on the outside of the two side walls 10, said side walls 10 can then be folded again into a closed position in which the housing 2 and the openings 7 thereof are closed, preferably in an airtight manner, with the limbs introduced therein. A configuration of this type of the therapy device 1 is particularly suitable for older people who are no longer very mobile. As previously stated, a respective additional opening 7, as indicated by a dash-dot line, can be provided, for example in the bottom of the housing 2 so that a foot, for example can be guided through in order to treat just the ankle. In this case, the support 19 can be omitted or can be provided to be removable. However, the additional opening 7 can be also provided in any other side of the housing 2, depending on the limb 4 to be treated.

Furthermore, the side walls 10 can optionally be respectively provided with at least one closure element (not shown), for example a catch for hooking or latching into the housing 2, or a magnetic lock etc. for holding the side walls 10 on the housing 2 in the closed or folded up position.

As previously stated with reference to FIG. 1, the openings 7 can be respectively provided with an additional seal for tightly sealing the openings 7 and the housing 2 after the limbs have been received in the housing 2. The cooling means 12 which is arranged, for example in the cold chamber 3 between the two limbs can optionally also be provided with or coupled to cooling ribs or cooling lamellae (not shown) on one or both sides. Furthermore, the cooling means 12, for example a Peltier element, can be fastened, for example screwed on in the housing 2, or can be provided to be replaceable. For this purpose, it is possible to provide in the housing 2 a mount (not shown) into which the cooling means 12, for example a metal plate, a freezer battery, a container with a cooling gel, ice cubes or a block of ice can be introduced.

As shown in the embodiment in FIG. 2, the ventilator means 17 are provided on the two side walls 10 of the housing 2. Likewise, the ventilator means 17 can be provided in any other place in the housing 2 to generate a cooling flow of air and/or to distribute the cool air generated by the cooling means 12. In this respect, the ventilator means 17 serves to generate the previously described wind chill effect over the limb or part or portion thereof which is to be treated. For example, at least one ventilator means 17 can be arranged on the bottom of the housing 2, on the top cover of the housing 2 or even on the cooling means 12 itself, etc. Furthermore, at least one guide plate (no shown) can optionally be provided to guide the air flow from the ventilator means 17.

As previously stated with reference to FIG. 1, in addition to a ventilator means 17 and/or a cooling means 12 it is also possible to provide at least one heating means for supplying heat into the cold chamber 3, if required.

Furthermore, the cooling means 12, the ventilator means 17 and/or the heating means, as previously stated, are configured to be controllable and/or regulatable, preferably by means of individual control means or by a common control means. In this respect, the cooling means 12, the ventilator means 17 and/or the heating means can be controlled and/or regulated, for example in respect of their actuation duration, subject to a temperature to be set in the cold chamber 3, subject to a temperature profile to be set in the cold chamber 3, etc. Furthermore, a temperature sensor means (not shown) for determining the temperature in the cold chamber 3 can be provided. The control means can control and/or regulate the cooling means 12, the ventilator means 17 and/or the heating means, subject to the temperature detected by the temperature sensor means. In this way, a constant, predetermined temperature or a predetermined temperature profile can be set in the cold chamber 3.

To enhance comfort and to obtain a position which is as painless as possible, a support 19, for example a lattice which allows the throughflow of air, is also provided in the cold chamber 3 for the particular limb. Furthermore, a rocker element 20 can optionally also be provided on the outside of the housing 2, on the bottom thereof. In the embodiment in FIG. 2, the rocker element 20 allows the therapy device 1 to rock backwards and forwards and can also be locked in position, if required.

As previously described with reference to FIG. 1, the housing 2 and the cold chamber 3 thereof can be configured, for, example with a bulge 11, as indicated by a dotted line in FIG. 2. The bulge 11 can provide an additional freedom of movement for a limb received in the therapy device 1.

FIG. 3 is a purely schematic and greatly simplified perspective view of a variant of the therapy device 1 of the second embodiment of the invention according to FIG. 2. The folding side walls 10 of the therapy device 1 are shown in an unfolded position, in which limbs such as feet can be easily introduced into the cold chamber 3 before the side walls 10 are then folded in and the housing 2 is closed, preferably in an airtight manner.

The variant of the therapy device 1 according to FIG. 3 substantially corresponds to the therapy device 1 according to FIG. 2, so that reference is made to the description of FIG. 2 to save unnecessary repetition.

The therapy device 1 according to FIG. 3 merely differs from the therapy device 1 according to FIG. 2 by the arrangement of the cooling means 12 on the inside of the top cover of the housing 2 and by the attachment of at least one ventilator means 17 to the cooling means 12. The cooling means 12 can be attached, for example screwed, onto the top cover of the housing 12, or can be received in a mount (not shown) of the housing 12 such that it can be replaced.

Due to the folding side walls 10, limbs such as feet can be easily introduced from the side, which is advantageous particularly for old people. Furthermore, using the strap 15 attached to the side walls 10, the side walls 10 can be easily returned to their closed position, without the person who is to be treated having to bend forwards in order to fold up the side walls 10. As previously stated, at least one closure element (not shown), for example a catch or a magnetic lock etc. can optionally also be provided for holding the side walls 10 in the closed or folded up position. Furthermore, as previously stated, the therapy device can also have at least one further opening 7 for passing a limb 4 through, to treat for example a part, such as a knee, an elbow, an ankle or wrist etc. of the limb 4. As indicated in FIG. 3 by a dash-dot line, the at least one additional opening 7 can be provided in the rear wall of the housing 2 opposite the top cover or adjacent thereto. Consequently, it is particularly easy, for example for a limb 4, for example an arm to pass through the housing 2 so that the elbow can be treated in the cold chamber 3 of the housing 2.

Although the present invention has been described above on the basis of the preferred embodiments, it is not restricted thereto, but can be modified in many different ways. In particular, the previously described embodiments can be combined together, particularly individual features thereof.

As stated previously by way of example with reference to FIGS. 1 to 3, the housing 2 of the therapy device 1 is preferably configured such that it is outwardly insulated. The housing 2 consists, for example of foamed polystyrene and/or of at least one plastics shell etc. In addition to foamed polystyrene and/or a plastics shell, it is possible to provide as insulation material any material permitted for devices of this type, including air for transparent wall parts for example or, for example also for inflatable wall parts etc. The advantage of foamed polystyrene and the plastics shell is that they are light and therefore it is possible to provide a therapy device 1 with the lowest weight possible. The invention is not restricted to the mentioned examples of foamed polystyrene and a plastics shell to form an insulated housing of the therapy device. Any other type of insulation can be provided which is suitable for providing a housing which is outwardly insulated. In this respect, the housing with its insulation is preferably of a particularly light configuration.

To supply power to electrically powered components of the therapy device, such as the ventilator means, an electrically powered cooling means, an electrically powered heating means, the control means etc., the therapy device can have at least one power plug, a power plug connection, an accumulator, a battery and/or at least one other suitable energy source to provide current.

### LIST OF REFERENCE NUMERALS

1 cold box, therapy device
2 housing
3 cold chamber
4 limb
5 person to be treated
6 hand
7 opening
8 cover
9 front side (housing)
10 side wall (housing)
11 bulge
12 cooling means
13 top cover
14 mount
15 strap
16 upper side (housing)
17 ventilator means, fan
18 guide plate
19 support
20 rocker element
21 longitudinal axis

1-20. (canceled)

21. A transportable therapy device for the therapeutic treatment of at least one person, in particular of a painful condition of the limb of the person, the transportable therapy device comprising:
a box-like housing which encloses at least one cold chamber which is only accessible from outside via at least one opening, wherein the at least one opening is formed and arranged in the housing in order to introduce a limb, which is to be treated, into the cold chamber,
at least one cooling means which is thermally coupled in such a way to the cold chamber to cool the cold chamber and is configured in such a way to generate in the cold chamber a predetermined temperature for the therapeutic treatment of the limb which has been introduced, for the therapeutic treatment of a painful condition of the limb which has been introduced.

22. The device of claim 21, wherein the cooling means has a fan to distribute and to cool by circulation the air in the cold chamber.

23. The device of claim 21, wherein at least one heating means is provided in the housing, which heating means is configured to supply heat to the cold chamber.

24. The device of claim 21, wherein a control means is provided which can control or regulate the cooling means, the fan or a heating means such that a predetermined temperature or a predetermined temperature profile can be set in the cold chamber, the predetermined temperature or the predetermined temperature profile on the skin being within a range of 15° C. to 25° C.

25. The device of claim 21, wherein a temperature sensor is provided for determining the temperature in the cold chamber.

26. The device of claim 21, wherein the cooling means contains at least one of:
at least one Peltier element,
at least one metal plate,
at least one freezer battery,
a cooling gel,
ice cubes;
at least one block of ice.

27. The device of claim 21, wherein the cooling means is configured with or coupled to cooling lamellae on at least a side facing the cold chamber.

28. The device of claim 21, wherein at least one guide plate is provided in the cold chamber of the housing for guiding a flow of air from the at least one fan.

29. The device of claim 21, wherein the opening has a seal or a cover which is configured and arranged in such a way to seal the opening when the limb has been introduced into the opening.

30. The device of claim 21, wherein at least one side wall is configured such that it folds open and is arranged in such a way to allow the limb to be introduced into the cold chamber of the housing when the side wall has been folded open.

31. The device of claim 21, wherein provided in the therapy device is a support which is arranged and positioned in the cold chamber in such a way to function as a supporting means for a limb introduced into the cold chamber.

32. The device of claim 21, wherein the housing has a mount for inserting the at least one cooling means, a top cover, a side wall or a bottom of the housing being removably configured in order to insert the at least one cooling means into the mount.

33. The device of claim 21, wherein the housing has at least two openings for guiding a respective limb through, the at least two openings being provided in at least one of:
in opposite sides of the housing,
in a side wall,
a front wall and a rear wall,
in a top cover,
in a bottom of the housing.

34. The device of claim 21, wherein an opening surface of an opening is respectively dimensioned in such a way to receive in the cold chamber of the housing a limb in the form of an upper arm, an elbow, a wrist, a foot, a shin or a knee.

35. The device of claim 21, wherein the housing is provided outside on the bottom thereof with a rocker element for rocking the therapy device from side to side.

36. The device of claim 21, wherein the housing and the cold chamber have a bulge for receiving the limb.

37. The device of claim 21, wherein the housing is configured to be outwardly insulated and it consists in particular of an insulation of, for example foamed polystyrene and/or at least one plastics shell.

38. The device of claim 21, wherein at least one top cover or at least one side wall of the housing is configured to be transparent.

39. The device of claim 21, wherein a wall of the top cover or of the side wall is configured in the interior thereof with a gaseous insulation.

40. The device of claim 21, wherein the flow of air generated by the at least one fan is strong enough to cool the limb, to be treated, by the wind chill effect, air close to the skin being removed convectively and, associated therewith, the evaporation rate being increased so that the equalisation of the surface temperature of the limb, received in the housing, with the ambient air temperature in the housing is accelerated.
